(19) United States
(12) Patent Application Publication
(10) Pub. No.: US 2018/0067181 A1
(43) Pub. Date: Mar. 8, 2018

(54) MAGNETIC FIELD ADJUSTING METHOD, MAGNETIC FIELD ADJUSTING APPARATUS, AND MAGNETIC RESONANCE IMAGING APPARATUS

(71) Applicant: Toshiba Medical Systems Corporation, Otawara-shi (JP)

(72) Inventors: Kazuto NOGAMI, Nasushiobara (JP); Fumitoshi KOJIMA, Otawara (JP); Yoshitomo SAKAKURA, Nasushiobara (JP); Hidekazu TANAKA, Nasushiobara (JP); Takehiro SHIBUYA, Nasushiobara (JP)

(73) Assignee: Toshiba Medical Systems Corporation, Otawara-shi (JP)

(21) Appl. No.: 15/696,849

(22) Filed: Sep. 6, 2017

(30) Foreign Application Priority Data
Sep. 7, 2016 (JP) 2016-174996

(51) Int. Cl. G01R 33/3873; G01R 33/38; G01R 33/389; A61B 5/055; H01F 7/18

(52) U.S. Cl. CPC G01R 33/3873; G01R 33/3802; H01F 7/1805; A61B 5/055; G01R 33/389

(57) ABSTRACT

A magnetic field adjusting method according to an embodiment includes: acquiring, by using a measuring device, first data related to a static magnetic field while a static magnetic field magnet is generating the static magnetic field having magnetic field intensity lower than rated magnetic field intensity required by an imaging process performed by a magnetic resonance imaging apparatus; and calculating, by using processing circuitry, a positional arrangement of a shim member used for correcting uniformity of the static magnetic field on the basis of the first data.

### CROSS-REFERENCE TO RELATED APPLICATIONS

This application is based upon and claims the benefit of priority from Japanese Patent Application No. 2016-174996, filed on Sep. 7, 2016; the entire contents of which are incorporated herein by reference.

### FIELD

Embodiments described herein relate generally to a magnetic field adjusting method, a magnetic field adjusting apparatus, and a magnetic resonance imaging apparatus.

### BACKGROUND

Examples of methods for performing a passive shimming process on a magnetic resonance imaging apparatus include a method by which shim members such as iron pieces are brought into a rated-intensity magnetic field, so that the shim members that were brought into the magnetic field are fixed onto the cylindrical inner wall surface of a magnet bore tube or the like, by using an adhesive agent or mechanical fastening members such as screws. According to another method, when the magnetic field intensity has been measured in a rated-intensity magnetic field, a demagnetization process is temporarily performed so that shim members are attached/detached and fixed in the absence of magnetic fields. After the shim members are attached/detached and fixed, a magnetized state is achieved again, up to the level of the rated-intensity magnetic field.

In the former example, however, in the rated-intensity magnetic field, the shim members are subject to a magnetic field attraction force from a static magnetic field generating device. Accordingly, it may take time to make necessary adjustments, for example. Further, when the shimming process is performed by using an adhesive agent, for example, it may be necessary, in some situations, to somehow hold and prevent the shim members from moving around (e.g., by pressing the shim members down with one's hands) until the adhesive agent becomes hard. In the latter example, a larger amount of refrigerant is consumed, for example. Also, because the latter example involves the demagnetization process, work hours of the user become longer, for example.

### BRIEF DESCRIPTION OF THE DRAWINGS

FIG. 1 is a diagram illustrating a magnetic resonance imaging apparatus according to an embodiment;

FIG. 2 is a flowchart illustrating a procedure in a shimming process performed by the magnetic resonance imaging apparatus according to the embodiment;

FIG. 3 is a flowchart illustrating details of the section at step S150 in FIG. 2 corresponding to a procedure in the shimming process performed by the magnetic resonance imaging apparatus according to the embodiment;

FIG. 4 is a chart for explaining a B-H curve of iron shims used in the shimming process performed by the magnetic resonance imaging apparatus according to the embodiment;

FIG. 5 is a drawing for explaining constituent elements of magnetic flux densities obtained at measuring points in relation to the shimming process performed by the magnetic resonance imaging apparatus according to the embodiment;

FIG. 6 is a diagram illustrating another example of an imaging process apparatus according to the embodiment; and

FIG. 7 is a diagram illustrating yet another example of the image processing apparatus according to the embodiment.

### DETAILED DESCRIPTION

A magnetic field adjusting method according to an embodiment includes: acquiring, by using a measuring device, first data related to a static magnetic field while a static magnetic field magnet is generating a static magnetic field having magnetic field intensity lower than rated magnetic field intensity required by an imaging process performed by a magnetic resonance imaging apparatus; and calculating, by using processing circuitry, a positional arrangement of a shim member used for correcting uniformity of the static magnetic field on the basis of the first data.

Exemplary embodiments of a magnetic resonance imaging apparatus will be explained in detail below, with reference to the accompanying drawings.

### Embodiments

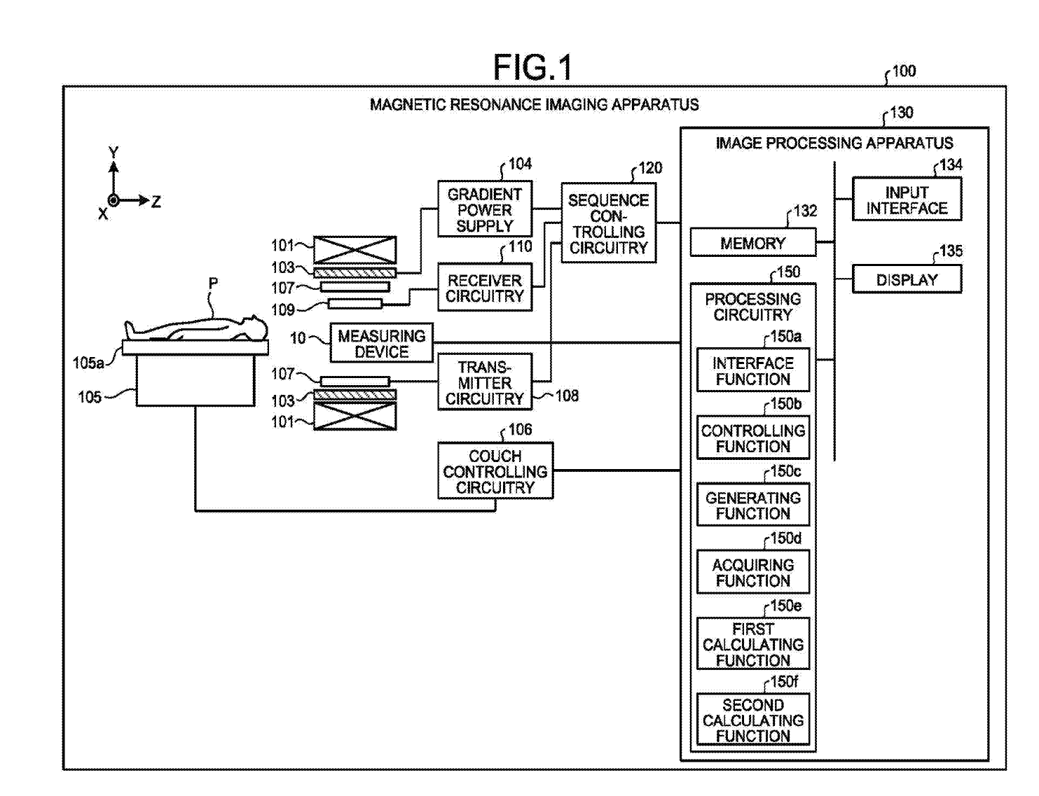

FIG. 1 is a block diagram illustrating a magnetic resonance imaging apparatus (hereinafter “MRI apparatus”) 100 according to an embodiment. As illustrated in FIG. 1, the MRI apparatus 100 includes a static magnetic field magnet 101, a gradient coil 103, a gradient power supply 104, a couch 105, couch controlling circuitry 106, a transmitter coil 107, transmitter circuitry 108, a receiver coil 109, receiver circuitry 110, sequence controlling circuitry 120, and an image processing apparatus 130. The MRI apparatus 100 does not include an examined subject (hereinafter, “patient”) P representing a human body, for example. Further, the configuration illustrated in FIG. 1 is merely an example. For instance, the sequence controlling circuitry 120 and any of the constituent elements of the image processing apparatus 130 may be integrated together or separated from the rest as appropriate.

The static magnetic field magnet 101 is a magnet formed to have a hollow and substantially circular cylindrical shape and is configured to generate a static magnetic field in the space on the inside thereof. For example, the static magnetic field magnet 101 may be realized with a superconductive magnet or the like and is configured to be magnetized by receiving a supply of an electric current from a static magnetic field power supply (not illustrated). The static magnetic field power supply is configured to supply the electric current to the static magnetic field magnet 101.

In place of the static magnetic field magnet 101, a permanent magnet may be used as a magnet. In that situation, the MRI apparatus 100 does not necessarily have to include the static magnetic field power supply. Further, the static magnetic field power supply may be provided separately from the MRI apparatus 100.

The gradient coil 103 is a coil formed to have a hollow and substantially circular cylindrical shape and is disposed on the inside of the static magnetic field magnet 101. The gradient coil 103 is formed by combining together three coils corresponding to X-, Y-, and Z-axes that are orthogonal to one another. These three coils are configured to individually receive the supply of the electric current from the gradient power supply 104 and to generate gradient magnetic fields of which the magnetic field intensities change along the X-, Y-, and Z-axes. The gradient magnetic fields along the X-, Y-, and Z-axes generated by the gradient coil 103 may be, for example, a slicing gradient magnetic field Gs, a phase-encoding gradient magnetic field Ge, and a read-out gradient magnetic field Gr. The gradient power supply 104 is configured to supply the electric current to the gradient coil 103.

The couch 105 includes a couchtop 105a on which the patient P is placed. Under control of the couch controlling circuitry 106, the couchtop 105a is inserted into the hollow space (an image taking opening) of the gradient coil 103, while the patient P is placed thereon. Usually, the couch 105 is installed in such a manner that the longitudinal direction thereof extends parallel to the central axis of the static magnetic field magnet 101. Under control of the image processing apparatus 130, the couch controlling circuitry 106 is configured to drive the couch 105 so as to move the couchtop 105a in the longitudinal direction and the up-and-down direction.

The transmitter coil 107 is disposed on the inside of the gradient coil 103 and is configured to generate a radio frequency magnetic field by receiving a supply of Radio Frequency (RF) pulse from the transmitter circuitry 108. The transmitter circuitry 108 is configured to supply the transmitter coil 107 with the RF pulse corresponding to a Larmor frequency determined by the type of the target atom and the magnetic field intensities.

The receiver coil 109 is disposed on the inside of the gradient coil 103 and is configured to receive magnetic resonance signals (hereinafter, “MR signals”) emitted from the patient P due to an influence of a radio frequency magnetic field. When having received the MR signals, the receiver coil 109 is configured to output the received MR signals to the receiver circuitry 110.

The transmitter coil 107 and the receiver coil 109 described above are merely examples. The coil structure may be configured by selecting one coil or combining two or more coils from among the following: a coil having only a transmitting function; a coil having only a receiving function; and a coil having a transmitting/receiving function.

The receiver circuitry 110 is configured to detect the MR signals output from the receiver coil 109 and to generate magnetic resonance data (hereinafter, “MR data”) on the basis of the detected MR signals. More specifically, the receiver circuitry 110 generates the MR data by applying a digital conversion to the MR signals output from the receiver coil 109. Further, the receiver circuitry 110 is configured to transmit the generated MR data to the sequence controlling circuitry 120. The receiver circuitry 110 may be provided on the gantry device side where the static magnetic field magnet 101, the gradient coil 103, and the like are provided.

The sequence controlling circuitry 120 is configured to perform an image taking process on the patient P, by driving the gradient power supply 104, the transmitter circuitry 108, and the receiver circuitry 110, on the basis of sequence information transmitted thereto from the image processing apparatus 130. In this situation, the sequence information is information defining a procedure to perform the image taking process. The sequence information defines: the intensity of the electric current supplied from the gradient power supply 104 to the gradient coil 103 and the timing with which the electric current is to be supplied; the intensity of the RF pulse supplied from the transmitter circuitry 108 to the transmitter coil 107 and the timing with which the RF pulse is to be applied; the timing with which the MR signals are to be detected by the receiver circuitry 110, and the like. For example, the sequence controlling circuitry 120 is configured with an integrated circuit such as an Application Specific Integrated Circuit (ASIC) or a Field Programmable Gate Array (FPGA), or an electronic circuit such as a Central Processing Unit (CPU) or a Micro Processing Unit (MPU).

When having received the MR data from the receiver circuitry 110 as a result of the image taking process performed on the patient P by driving the gradient power supply 104, the transmitter circuitry 108, and the receiver circuitry 110, the sequence controlling circuitry 120 transfers the received MR data to the image processing apparatus 130.

The image processing apparatus 130 is configured to exercise overall control of the MRI apparatus 100 and to generate images and the like. The image processing apparatus 130 includes a memory 132, an input interface 134, a display 135, and processing circuitry 150. The processing circuitry 150 includes an interface function 150a, a controlling function 150b, a generating function 150c, an acquiring function 150d, a first calculating function 150e, and a second calculating function 150f.

In an embodiment, processing functions implemented by the interface function 150a, the controlling function 150b, the generating function 150c, the acquiring function 150d, the first calculating function 150e, and the second calculating function 150f are stored in the memory 132 in the form of computer-executable programs. The processing circuitry 150 is a processor configured to realize the functions corresponding to the computer programs (hereinafter, “programs”) by reading the programs from the memory 132 and executing the read programs. In other words, the processing circuitry 150 that has read the programs has the functions illustrated within the processing circuitry 150 in FIG. 1. Although FIG. 1 illustrates the example in which the single processing circuitry 150 realizes the processing functions implemented by the interface function 150a, the controlling function 150b, the generating function 150c, the acquiring function 150d, the first calculating function 150e, and the second calculating function 150f, another arrangement is also acceptable in which the processing circuitry 150 is structured by combining together a plurality of independent processors so that the functions are realized as a result of the processors executing the programs.

In other words, each of the abovementioned functions may be structured as a program so that the single processing circuitry executes the programs. Alternatively, specific one or more of the functions may be installed in each of the dedicated and independent program-executing circuits.

The term “processor” used in the above explanation denotes, for example, a Central Processing Unit (CPU), a Graphical Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions thereof by reading and executing the programs stored in the memory 132.

The acquiring function 150d, the first calculating function 150e, and the second calculating function 150f are examples of an acquiring unit, a first calculating unit, and a second calculating unit, respectively.

Instead of storing the programs into the memory 132, it is also acceptable to directly incorporate the programs into the circuits of the one or more processors. In that situation, the one or more processors realize the functions thereof by reading and executing the programs incorporated in the circuit thereof. Similarly, the couch controlling circuitry 106, the transmitter circuitry 108, the receiver circuitry 110, and the like are also each configured with an electronic circuit such as the processor described above.

By employing the interface function 150a, the processing circuitry 150 is configured to transmit the sequence information to the sequence controlling circuitry 120 and to receive the MR data from the sequence controlling circuitry 120. Further, when having received the MR data, the processing circuitry 150, which includes the interface function 150a, stores the received MR data into the memory 132. The MR data stored in the memory 132 is arranged into a k-space by the controlling function 150b. As a result, the memory 132 stores therein k-space data.

The memory 132 stores therein the MR data received by the processing circuitry 150 including the interface function 150a, the k-space data arranged in the k-space by the processing circuitry 150 including the controlling function 150b, image data generated by the processing circuitry 150 including the generating function 150c, and the like. For example, the memory 132 is configured by using a semiconductor memory element such as a Random Access Memory (RAM) or a flash memory, a hard disk, an optical disk, or the like.

The input interface 134 is configured to receive various types of instructions and inputs of information from the operator. For example, the input interface 134 is configured with a pointing device such as a mouse or a trackball, a selecting device such as a mode changing switch, and/or an input device such as a keyboard. Under control of the processing circuitry 150 including the controlling function 150b, the display 135 is configured to display a Graphical User Interface (GUI) used for receiving inputs of image taking conditions, as well as images and the like generated by the processing circuitry 150 including the generating function 150c. The display 135 may be, for example, a display device such as a liquid crystal display monitor.

By employing the controlling function 150b, the processing circuitry 150 is configured to exercise overall control of the MRI apparatus 100 to control image taking processes, image generating processes, image displaying processes, and the like. For example, the processing circuitry 150 including the controlling function 150b receives an input of an image taking condition (e.g., an image taking parameter) via the GUI and generates the sequence information according to the received image taking condition. Further, the processing circuitry 150 including the controlling function 150b transmits the generated sequence information to the sequence controlling circuitry 120.

By employing the generating function 150c, the processing circuitry 150 reads the k-space data from the memory 132 and generates an image by performing a reconstructing process such as a Fourier transform on the read k-space data.

Further, the processing circuitry 150 includes various types of functions such as the acquiring function 150d, the first calculating function 150e, and the second calculating function 150f. These functions will be explained later.

A measuring device 10 is a measuring device configured to measure intensities of the magnetic field. The measuring device 10 is configured to measure the magnetic field in various locations, while the static magnetic field magnet 101 is generating rated magnetic field intensity required by imaging processes performed by the MRI apparatus and is configured to measure the magnetic field in various locations, while the static magnetic field magnet 101 is generating a static magnetic field having magnetic field intensity lower than the rated magnetic field intensity. For example, the measuring device 10 is configured by using one or more Nuclear Magnetic Resonance (NMR) probes. In one example, the measuring device 10 is configured to have a spherical shape by using a plurality of NMR probes. Alternatively, the measuring device 10 may be configured by using a single NMR probe, so as to sequentially measure magnetic field intensities of a plurality of points.

The data measured by the measuring device 10 is sent to the image processing apparatus 130 connected to the measuring device 10 and is used for data processing purposes.

In this situation, the measuring device 10 may be configured as a part of the MRI apparatus 100. However, the measuring device 10 does not necessarily have to be included in the MRI apparatus 100.

The MRI apparatus 100 according to the embodiment is subject to a shimming process. The shimming process is an adjusting operation to correct spatial non-uniformity of the static magnetic field generated by the static magnetic field magnet 101 or the like included in the MRI apparatus 100, so as to improve uniformity. Typically, the shimming process is performed when the MRI apparatus 100 is installed. In the present example, for the MRI apparatus 100, the shimming process is performed by performing a passive shimming process, for instance. In this situation, the passive shimming process is, for example, a shimming process by which the static magnetic field in an image taking region is made uniform by arranging shim members (iron pieces) or the like in the static magnetic field generated by the static magnetic field magnet 101 or the like. As an example of the passive shimming process, a method is known by which very small passive shim members such as iron pieces (not illustrated) are fixed onto the cylindrical inner wall surface made of a magnet or the like, by using an adhesive agent or mechanical fastening members such as screws. As another example of the passive shimming process, another method is also known by which passive shim members are fixed by being inserted into a shim member fixing component part (e.g., a shim tray (not illustrated) used for fixing the shim members into certain positions) used for attachment of the passive shim members. As yet another example of the shimming process, for the MRI apparatus 100, a shimming process may be performed by performing an activating shimming process, for instance. In this situation, the active shimming process is, for example, a shimming process by which the static magnetic field in an image taking region is made uniform, by causing an electric current to flow through a shim coil (not illustrated) and using the magnetic field generated as a result of the electric current flowing through the shim coil.

Next, a background of the embodiment will briefly be explained.

Examples of methods for performing the passive shimming process on an MRI apparatus include a method by which shim members such as iron pieces are brought into a rated-intensity magnetic field, so that the shim members that were brought into the magnetic field are fixed onto the cylindrical inner wall surface of a magnet bore tube or the like, by using an adhesive agent or mechanical fastening members such as screws. According to another method, when the magnetic field intensity has been measured in a rated-intensity magnetic field, a demagnetization process is temporarily performed so that shim members are attached/detached and fixed in the absence of magnetic fields. After the shim members are attached/detached and fixed, a magnetized state is achieved again, up to the level of the rated-intensity magnetic field.

In the former example, however, in the rated-intensity magnetic field, the shim members are subject to a magnetic field attraction force or the like from a static magnetic field generating device. Accordingly, it may take time to make necessary adjustments, for example. Further, when the shimming process is performed by using an adhesive agent, for example, it may be necessary, in some situations, to hold and prevent the shim members from moving around (by pressing the shim members down with one's hands) until the adhesive agent becomes hard. In the latter example, a larger amount of refrigerant is consumed, for example. Also, because the latter example involves the demagnetization process, work hours of the user become longer, for example.

Consequently, it is desirable to perform the shimming process in a low-intensity magnetic field having a magnetic field intensity lower than that of the rated-intensity magnetic field. In view of this background, the MRI apparatus 100 according to the embodiment includes the acquiring function 150d, the first calculating function 150e, and the second calculating function 150f. In this situation, by employing the acquiring function 150d, the processing circuitry 150 measures a magnetic flux density in a low-intensity magnetic field. More specifically, the measuring device 10 measures the magnetic flux density in the low-intensity magnetic field and transmits the measured result to the processing circuitry 150. By employing the first calculating function 150e, the processing circuitry 150 calculates a magnetic flux density in the rated-intensity magnetic field on the basis of the magnetic flux density in the low-intensity magnetic field. Further, by employing the second calculating function 150f, the processing circuitry 150 calculates a positional arrangement of the shim members on the basis of the calculated magnetic flux density.

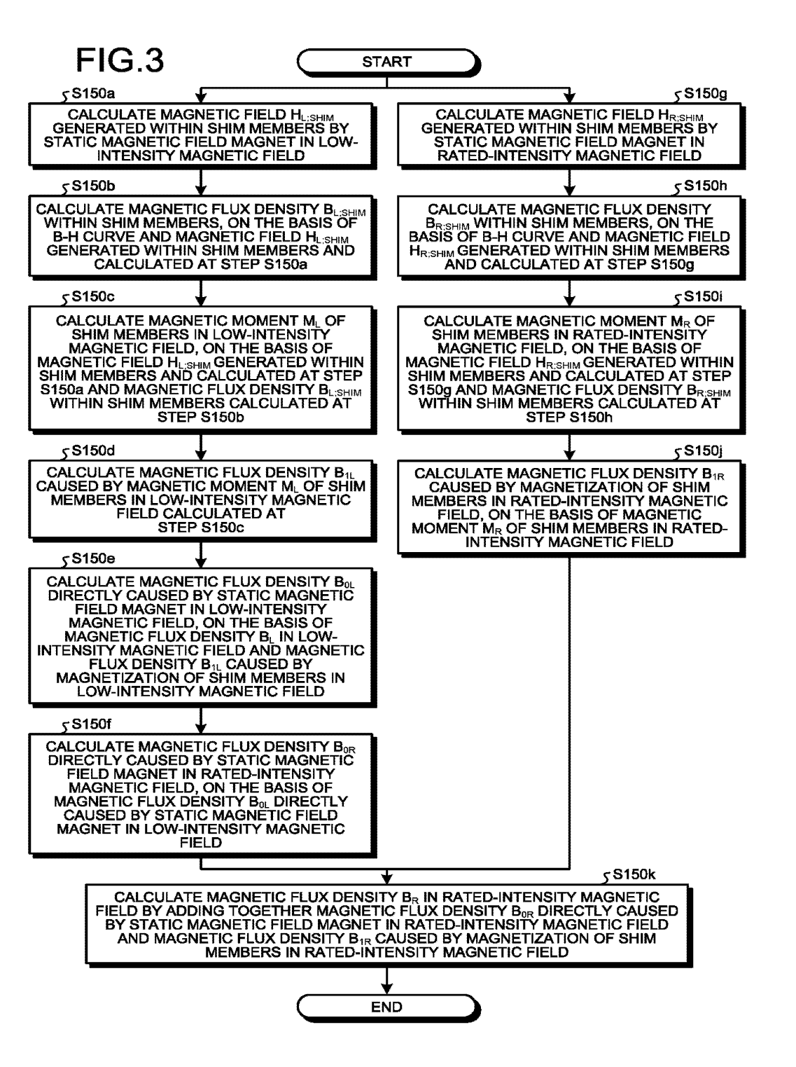

When the B-H curve of the shim members exhibits a non-linear behavior, it is desirable to configure the first calculating function to perform the step of calculating the magnetic flux density in the rated-intensity magnetic field on the basis of the magnetic flux density in the low-intensity magnetic field, in consideration of the non-linearity of the B-H curve of the shim members. In that situation, by performing the process explained below with reference to FIG. 3, the MRI apparatus 100 according to the embodiment is able to calculate an estimated value of the magnetic flux density in the rated-intensity magnetic field on the basis of the magnetic flux density in the low-intensity magnetic field, even when the B-H curve of the shim members exhibits a non-linear behavior.

The MRI apparatus 100 according to the embodiment configured as described above is able to decrease the work hours of the user or the like, when it is possible, for example, to perform the shimming process in two steps, namely the magnetization up to the level of the low-intensity magnetic field and the magnetization up to the level of the rated-intensity magnetic field, in place of the step of repeatedly performing the magnetization and the demagnetization. In addition, with this arrangement, it is also possible to decrease the consumption amount of the refrigerant such as liquid helium, for example.

For example, the effect of improving the refrigerant consumption amount is proportional to (the magnetic field intensity of the low-intensity magnetic field/the magnetic field intensity of the rated-intensity magnetic field))² For example, when the magnetization is performed in a low-intensity magnetic field of which the magnetic field intensity is 50% of that of the rated-intensity magnetic field, the refrigerant consumption amount is equal to 25% of that in the example with the rated-intensity magnetization. Further, for example, the work hours of the user are proportional to (the magnetic field intensity of the low-intensity magnetic field/the magnetic field intensity of the rated-intensity magnetic field). For example, when the magnetization is performed in a low-intensity magnetic field of which the magnetic field intensity is 50% of that of the rated-intensity magnetic field, the work hours of the user are equal to 50% of those in the example with the rated-intensity magnetization.

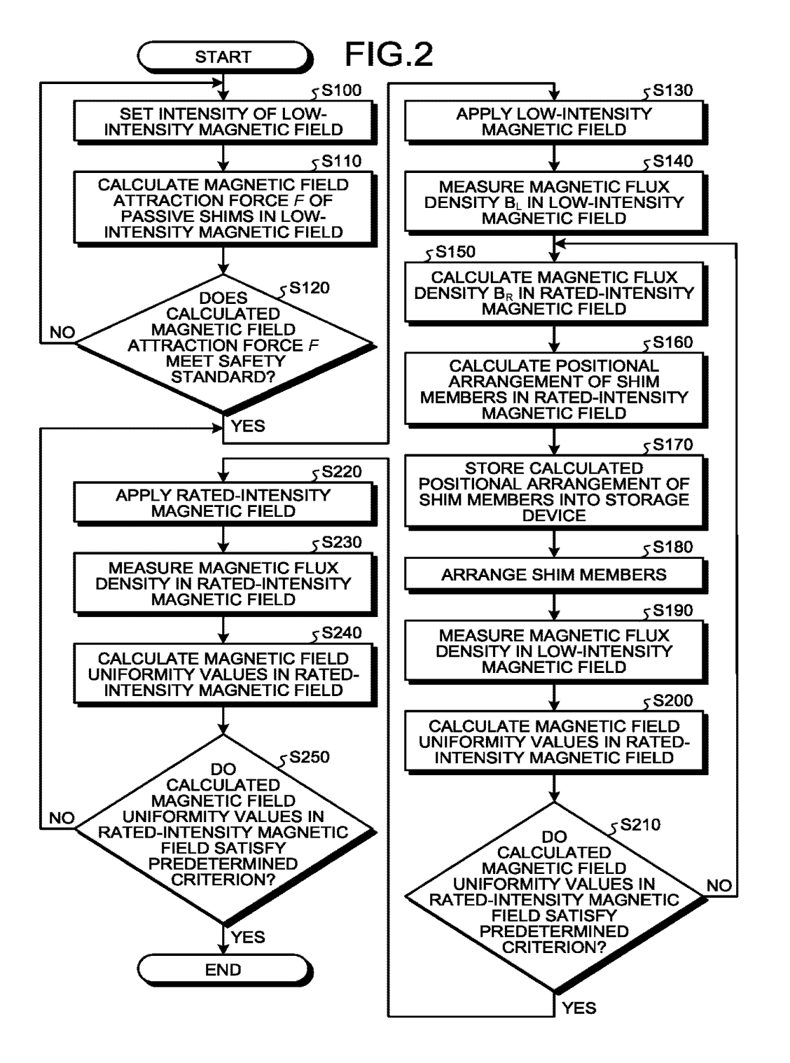

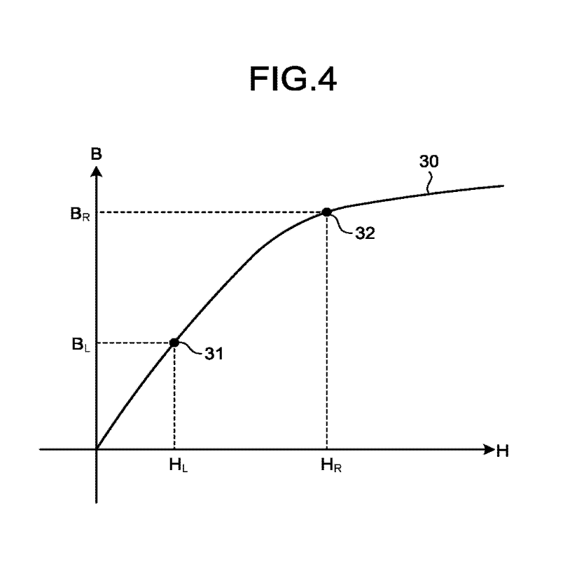

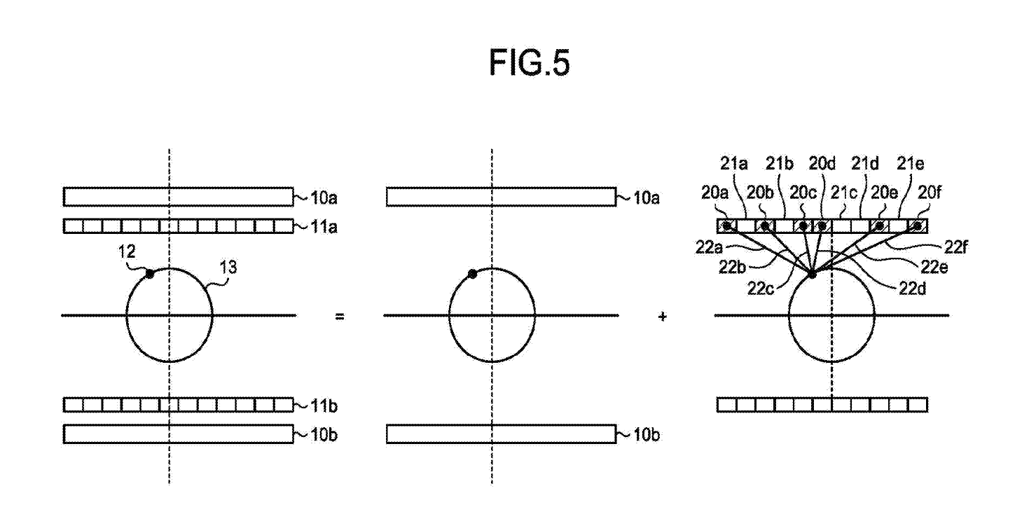

Next, details of processes according to the embodiment will be explained, with reference to FIGS. 2 to 5. FIGS. 2 and 3 are flowcharts illustrating a shimming procedure in a process performed by the MRI apparatus 100 according to the embodiment. FIG. 2 is a flowchart illustrating the entirety of the shimming process performed by the MRI apparatus 100. In contrast, FIG. 3 is a flowchart illustrating details of step S150 in FIG. 2. FIGS. 4 and 5 are drawings for explaining processes performed by the MRI apparatus according to the embodiment.

First, by employing the controlling function 150b, the processing circuitry 150 sets the intensity of the low-intensity magnetic field applied at step S130 (step S100). For example, the processing circuitry 150 receives, via the input interface 134, an input regarding the intensity of the static magnetic field applied by the static magnetic field power supply at step S130 and sets the received value as an approximate value of the intensity of the static magnetic field applied by the static magnetic field power supply at step S130. In this situation, the intensity of the static magnetic field applied by the static magnetic field power supply is kept in correspondence with the value of the electric current caused by the static magnetic field power supply to flow through the static magnetic field magnet 101. Accordingly, setting the intensity value of the static magnetic field applied by the static magnetic field power supply denotes, for example, setting the value of the electric current caused by the static magnetic field power supply to flow through the static magnetic field magnet 101.

In the following sections, when necessary (e.g., within substances), a magnetic field H and a magnetic flux density B will be differentiated from each other. In the present example, the magnetic flux density B denotes a variable that, together with an electric field E, structures a Maxwell equation in vacuum. In contrast, the magnetic field H denotes a variable that, together with an electric flux density D, structures a Maxwell equation in substances.

In the present example, in vacuum, the magnetic flux density B is expressed as B=μ₀H, by using H representing the magnetic field and μ₀ serving as a constant representing the magnetic permeability in vacuum. In contrast, in substances such as in shim members, the magnetic flux density B is expressed with Expression (1) below, by using H representing the magnetic field and μ representing the magnetic permeability unique to the substances.

$$\begin{matrix}\begin{matrix}B = µ H \\ = {µ}_{0} H + M\end{matrix} & ( 1 )\end{matrix}$$

In this situation, the magnetic moment M denotes the magnitude of a magnetic moment occurring in the substances due to the magnetic field H. For substances other than ferromagnetic substances (e.g., iron pieces), the magnetic moment M exhibits a smaller value. In contrast, for ferromagnetic substances (e.g., iron pieces used as the shim members), the magnetic moment M exhibits a larger value. As a result of the occurrence of the magnetic moment M, a new magnetic field is generated on the outside of the system.

It is possible to express the magnetic moment M by using Expression (2) below, which is obtained by deforming Expression (1).

M=B−μ₀H (2)

Expression (1) indicates that, as a result of the magnetic moment M being induced in the substances by the magnetic field H formed by the external magnetic field applied from the outside, the sum of a magnetic field (an internal magnetic field) newly generated on the outside of the system and the magnetic field H formed by the external magnetic field originally applied from the outside is equal to the magnetic flux density B. In substances, because the magnetic flux density B and the magnetic field H are in a proportional relationship, μ representing a factor of proportionality is referred to as the magnetic permeability. The magnetic permeability μ is dependent on the magnetic field H, when the B-H curve is non-linear, for example.

In the following explanations, in substances (e.g., within the shim members), the magnetic flux density B and the magnetic field H will be differentiated from each other. In contrast, in vacuum, because measuring of the magnetic flux density B is substantially equivalent to measuring of the magnetic field H, both the measuring of the magnetic flux density B and the measuring of the magnetic field H will simply be referred to as “measuring the magnetic field”.

In other words, the “intensity” of the low-intensity magnetic field at step S100, for example, denotes the “intensity of the magnetic field” in a general sense and signifies the magnitude of the magnetic flux density B, for example. However, possible embodiments are not limited to this example. It is also acceptable to interpret that the “intensity” of the low-intensity magnetic field denotes the intensity of the magnetic field H.

After that, the processing circuitry 150 calculates the value of a magnetic field attraction force F(B_{0L}) of the passive shim members corresponding to the intensity (the magnetic flux density B_{0L}) of the low-intensity magnetic field set at step S100 (step S110). In this situation, the low-intensity magnetic field denotes a magnetic field having an intensity lower than that of the rated-intensity magnetic field. Subsequently, the processing circuitry 150 judges whether or not the magnetic field attraction force F(B_{0L}) calculated at step S110 meets a safety standard (step S120). When the processing circuitry 150 determines that the magnetic field attraction force F(B_{0L}) calculated at step S110 meets the safety standard (step S120: Yes), the process proceeds to step S130. On the contrary, when the processing circuitry 150 determines that the magnetic field attraction force F(B_{0L}) calculated at step S110 does not meet the safety standard (step S120: No), the process returns to step S100, where the setting of the intensity of the low-intensity magnetic field applied at step S130 is reconsidered. More specifically, in that situation, the processing circuitry 150 sets a magnetic field intensity value that is smaller than the initially-set magnetic field intensity value, as the intensity of the low-intensity magnetic field. After that, the processing circuitry 150 judges again whether or not the new value meets the safety standard.

After that, the static magnetic field power supply applies a low-intensity magnetic field having the value set at step S100 (step S130). More specifically, the static magnetic field power supply causes an electric current having a first electric current value (hereinafter, simply “first current value”) I_{L} to flow through the static magnetic field magnet 101, the first current value I_{L} being a current value corresponding to the low-intensity magnetic field. In the following sections, an example will be explained in which the shim members are arranged with a first positional arrangement, for example, which is a predetermined positional arrangement. In the present example, the first positional arrangement is an initial positional arrangement used at the time of the installation of the MRI apparatus 100, for example.

Subsequently, by employing the receiver circuitry 110 and the measuring device 10, for example, the MRI apparatus 100 measures the value of a first magnetic flux density B_{L} representing the magnetic flux density in the low-intensity magnetic field (step S140). In other words, by employing the acquiring function 150d, the processing circuitry 150 acquires first data related to the first magnetic flux density B_{L}. The first magnetic flux density B_{L} is the magnetic flux density in the situation where the shim members are arranged with the first positional arrangement that is the predetermined positional arrangement, while the electric current having the first current value I_{L} corresponding to the low-intensity magnetic field is flowing through the static magnetic field magnet 101. In other words, the measuring device 10 acquires the first data related to the static magnetic field while the static magnetic field magnet is generating the static magnetic field having intensity lower than the rated magnetic field intensity required by imaging processes performed by the MRI apparatus 100. The measuring device 10 then transmits the acquired first data to the processing circuitry 150. In this situation, the first data is data that is related to the magnetic flux density.

In this situation, the first magnetic flux density B_{L} acquired at step S140 is approximately equal to the magnetic flux density B_{0L} directly caused by the electric current that has the first current value I_{L} flowing through the static magnetic field magnet 101. However, the first magnetic flux density B_{L} is different from the magnetic flux density B_{0L} by an amount corresponding to a magnetic flux density B_{1L} generated by the magnetic moment M_{L} in the surrounding of the shim members, the magnet moment M_{L} occurring in the shim members as a result of the electric current that has the first current value I_{L} flowing through the static magnetic field magnet 101. More specifically, when the position in which the first magnetic flux density B_{L} is measured is expressed as y, it is possible to express the value of the first magnetic flux density B_{L} in the measuring position y by using Expression (3) below.

B_{L}(y)=B_{0L}(y)+B_{1L}(y) (3)

In Expression (3), the element B_{L}(y) denotes a measured value representing the first magnetic flux density B_{L} in the measuring position y. Further, the element B_{0L}(y) denotes the magnetic flux density B_{0L} in the measuring position y that is considered to be directly caused by the electric current that has the first current value I_{L} flowing through the static magnetic field magnet 101. Further, the element B_{1L}(y) denotes the magnetic flux density B_{1L} in the measuring position y generated by the magnetic moment M_{L} in the surroundings of the shim member, the magnetic moment M_{L} occurring in the shim member due to the electric current that has the first current value I_{L} flowing through the static magnetic field magnet 101. Assuming that no shim member is present, the processing circuitry 150 estimates the value of the magnetic flux density B_{0L} that is considered to be directly caused by the electric current that has the first current value I_{L} flowing through the static magnetic field magnet 101. As explained later, it is possible to calculate the value of the magnetic flux density B_{0L} by subtracting the calculated value of B_{1L}(y) from B_{L}(y) measured by the measuring device 10. On the basis of the estimated value of the magnetic flux density B_{0L}, the processing circuitry 150 estimates the value of a magnetic flux density B_{0R} that is considered to be directly caused by an electric current that has a second current value I_{R} flowing through the static magnetic field magnet 101. On the basis of the estimated value of the magnetic flux density B_{0R}, the processing circuitry 150 further calculates the value of a second magnetic flux density B_{R} representing the magnetic flux density in the rated-intensity magnetic field. In this situation, the measuring position y symbolically expresses the position vector of a three-dimensional position, for example, and is characterized with a set of polar coordinates such as (r,θ,φ) expressed by using the center of the magnetic field as the origin, for example.

After that, by employing the first calculating function 150e, the processing circuitry 150 calculates the value of the second magnetic flux density B_{R} representing the magnetic flux density in the rated-intensity magnetic field (step S150). More specifically, by employing the first calculating function 150e, the processing circuitry 150 calculates, on the basis of the first data, the value of the second magnetic flux density B_{R} in the situation where the shim members are arranged with the first positional arrangement while the electric current having the second current value I_{R} larger than the first current value I_{L} is flowing through the static magnetic field magnet 101.

In this situation, when the B-H curve of the shim members has a linear characteristic, the magnetic flux density and the electric current are in a proportional relationship with each other. The processing circuitry 150 is therefore able to easily calculate the value of the second magnetic flux density B_{R}. In that situation, by employing the first calculating function 150e, the processing circuitry 150 calculates, on the basis of the first data, the value of the second magnetic flux density B_{R} in the situation where the electric current having the second current value I_{R} larger than the first current value I_{L} is flowing through the static magnetic field magnet 101, by using the expression B_{R}=I_{R}/I_{L}×B_{L}, for example.

However, when the B-H curve of the shim members has a non-linear characteristic, the total magnetic flux density and the electric current are not simply in a proportional relationship with each other. It is therefore necessary to use a special method for calculating the second magnetic flux density B_{R}. In view of these circumstances in the background, by employing the first calculating function 150e, the processing circuitry 150 calculates, at step S150, the value of the second magnetic flux density B_{R}, on the basis of the first data and a magnetic characteristic of the shim members. In this situation, the magnetic characteristic of the shim members may be, for example, a correspondence relationship between the magnitude of the magnetic field H applied to the shim members and the magnitude of the magnetic flux density B caused on the shim members. In other words, by employing the first calculating function 150e, the processing circuitry 150 calculates, on the basis of the first data, second data related to a static magnetic field in the situation where the static magnetic field magnet 101 is generating the static magnetic field having the rated intensity, by using a magnetization curve of substances contained in the shim members.

Details of the above process will be explained with reference to the flowchart in FIG. 3 and FIGS. 4 and 5. The flowchart in FIG. 3 is a flowchart illustrating the details of the process at step S150 in FIG. 2.

First, by employing the first calculating function 150e, the processing circuitry 150 calculates, on the basis of the first current value I_{L}, a magnetic field H_{L;shim} generated within the shim members by the static magnetic field magnet 101 in the low-intensity magnetic field (step S150a). More specifically, by employing the first calculating function 150e, the processing circuitry 150 calculates, on the basis of the first current value I_{L}, the magnetic field H_{L;shim} generated within the shim members, by using the Ampere's Law, the Biot-Savart Law, or the like.

Subsequently, by employing the first calculating function 150e, the processing circuitry 150 calculates a magnetic flux density B_{L;shim} within the shim members, on the basis of the magnetic characteristic of the shim members and the magnetic field H_{L;shim} generated in the shim members and calculated at step S150a (step S150b). In this situation, the magnetic characteristic may be, for example, the correspondence relationship, which is a so-called B-H curve, between the magnitude of the magnetic field H applied to the shim members and the magnitude of the magnetic flux density B caused on the shim members. The B-H curve is a curve unique to each substance. For example, when the material and the like of the shim members are the same as one another, the curve will be the same. Accordingly, by performing a predetermined measuring process in advance, for example, the processing circuitry 150 is able to obtain the B-H curve of the shim members in advance. Further, in another example, the processing circuitry 150 is able to obtain the B-H curve of the shim members in advance, by referring to a predetermined database, for instance. The image processing apparatus 130 may store the B-H curve of the shim members obtained in advance in this manner, into the memory 132, for example. In that situation, the processing circuitry 150 acquires the B-H curve of the shim members stored in the memory 132 by employing the acquiring function 150d, for example.

FIG. 4 illustrates an example of the B-H curve of the shim members. The horizontal axis expresses the magnetic field H applied to the inside of the shim members. The vertical axis expresses the magnetic flux density B caused on the shim members. The B-H curve 30 is the B-H curve of the shim members. The point 31 indicates a position on the B-H curve 30 in the low-intensity magnetic field. The point 32 indicates a position on the B-H curve 30 in the rated-intensity magnetic field. In FIG. 4, the magnetic field H_{L} indicates the magnetic field generated within the shim members in the low-intensity magnetic field and denotes the magnetic field H_{L;shim} calculated at step S150a. The first magnetic flux density B_{L} indicates the magnetic flux density within the shim members in the low-intensity magnetic field and corresponds to the magnetic flux density B_{L;shim} calculated at step S150b. The magnetic field H_{R} indicates the magnetic field generated within the shim members in the rated-intensity magnetic field and denotes the magnetic field H_{R;shim} calculated at step S150g. The magnetic flux density B_{R} indicates the magnetic flux density within the shim members in the rated-intensity magnetic field and corresponds to the magnetic flux density B_{R;shim} calculated at step S150h.

As explained above, at step S150b, by employing the first calculating function 150e, the processing circuitry 150 calculates the point 31 that is the intersection point of the value (H_{L}) of the magnetic field H_{L;shim} generated within the shim members and calculated at step S150a and the B-H curve 30 obtained from the memory 132 and further calculates the magnetic flux density B_{L;shim} within the shim members on the basis of the value B_{L} of the calculated point 31 on the vertical axis.

Possible examples of the magnetic characteristic of the shim members are not limited to the example described above. For instance, the magnetic characteristic of the shim members may be the magnetic permeability μ of the shim members expressed as a mathematical function of at least one selected from between the magnetic field H and the magnetic flux density B. By employing the first calculating function 150e, the processing circuitry 150 is able to calculate the magnetic flux density B_{L;shim} within the shim members on the basis of the magnetic field H_{L;shim} within the shim members and the magnetic permeability (H) (or (B)), by evaluating the right-hand side of Expression (1).

After that, by employing the first calculating function 150e, the processing circuitry 150 calculates a magnetic moment M_{L} of the shim members in the low-intensity magnetic field, on the basis of the magnetic field H_{L;shim} generated within the shim members and the magnetic flux density B_{L;shim} within the shim members (step S150c). For example, by employing the first calculating function 150e, the processing circuitry 150 calculates the magnetic moment M_{L} of the shim members in the low-intensity magnetic field, by calculating M_{L}=B_{L}−μ₀H_{L} according to Expression (2).

In this situation, a method for calculating the magnetic flux density B generated in a predetermined position r by a given magnetic moment m is already known. In actuality, except for prescribed constant multiplications, it is possible to express the magnetic flux density B as −grad(m·r/r₀³). In this expression, the letter m denotes a three-dimensional vector representing the magnetic moment; the letter r denotes a three-dimensional position vector expressed while the position of the magnetic moment is used as the origin; and the element r₀ denotes a scalar quantity expressing the magnitude of r.

In other words, on the basis of the value of the magnetic moment M_{L} of the shim members in the low-intensity magnetic field calculated at step S150c, the processing circuitry 150 calculates the value of the magnetic flux density B_{1L} caused by the magnetic moment M_{L} in the measuring position of the magnetic flux density (step S150d).

The situation described above can be expressed by using Expression (4) below, for example.

B_{1L}(y)=∫d×M_{L}(x)R(x,y) (4)

In Expression (4), the letter y denotes the measuring position, while the letter x denotes the position of the shim member. The element B_{1L}(y) denotes the value of the magnetic flux density in the measuring position y calculated at step S150d. The element M_{L}(x) denotes the value of the magnetic moment M_{L} in the position x calculated at step S150c. The coefficient R (x,y) is information indicating the magnitude of the contribution made by the magnetic moment M_{L}(x) in the position x to the magnetic flux density B_{1L}(y) in the measuring position y.

FIG. 5 illustrates the situations in Expressions (3) and (4). The coil 10a and the coil 10b represent the static magnetic field magnet 101. The shim tray 11a represents a shim tray in which the shim members are disposed. The measuring position 12 represents the measuring position in which the first magnetic flux density B_{L} is measured at step S140. The shim members 20a, 20b, 20c, 20d, 20e, and 20f represent regions in the shim tray 11a into which ferromagnetic members serving as the shim members are inserted. In contrast, the regions 21a, 21b, 21c, 21d, and 21e represent regions in the shim tray 11a into which no ferromagnetic members serving as the shim members are inserted. The straight line 22a is a straight line connecting the shim member 20a to the measuring position 12. Similarly, the straight lines 22b, 22c, 22d, 22e, and 22f are straight lines connecting the shim members 20b, 20c, 20d, 20e, and 20f to the measuring position 12, respectively.

The left section of FIG. 5 illustrates the element B_{L}(y) in Expression (3), i.e., the first magnetic flux density B_{L} in the measuring position y. The middle section of FIG. 5 illustrates the element B_{0L}(y) in Expression (3), i.e., the third magnetic flux density B_{0L} representing the magnetic flux density in the measuring position y directly caused by the electric current that has the first current value I_{L} flowing through the coils 10a and 10b. The right section of FIG. 5 illustrates the magnetic flux density B_{1L}(y) in Expression (3), i.e., the magnetic flux density B_{1L} in the measuring position y generated in the surroundings of the shim members by the magnetic moment M_{L} occurring in the shim members 20a, 20b, 20c, 20d, 20e, and 20f, as a result of an electric current that has the first current value I_{L} flowing through the static magnetic field magnet 101.

As indicated in Expression (4), the magnetic flux density B_{1L}(y) is obtained by integrating the product of the term of the magnetic moment M_{L}(x) occurring in the shim member and the information R(x,y) related to the positional arrangement of the shim member, with respect to the position x of the shim member. In the example in the right section of FIG. 5, for instance, the magnetic flux density B_{1L} in the measuring position 12 is calculated as the sum of the following products: the product of the magnetic moment occurring in the shim member 20a and a coefficient calculated on the basis of the length and the direction of the straight line 22a; the product of the magnetic moment occurring in the shim member 20b and a coefficient calculated on the basis of the length and the direction of the straight line 22b; the product of the magnetic moment occurring in the shim member 20c and a coefficient calculated on the basis of the length and the direction of the straight line 22c; the product of the magnetic moment occurring in the shim member 20d and a coefficient calculated on the basis of the length and the direction of the straight line 22d; the product of the magnetic moment occurring in the shim member 20e and a coefficient calculated on the basis of the length and the direction of the straight line 22e; and the product of the magnetic moment occurring in the shim member 20f and a coefficient calculated on the basis of the length and the direction of the straight line 22f. In this situation, the regions 21a, 21b, 21c, 21d, and 21e are regions into which no ferromagnetic members serving as the shim members are inserted. Accordingly, because the magnetic moment in each of these regions is substantially zero, the contribution made by each of these regions to the magnetic flux density B_{1L} is substantially equal to zero.

After that, the processing circuitry 150 calculates the value of the magnetic flux density B_{0L} that is considered to be directly caused by the electric current that has the first current value I_{L} flowing through the static magnetic field magnet 101 in the low-intensity magnetic field, by using the magnetic flux density B_{L}(y) in the low-intensity magnetic field represented by the measured value obtained by the measuring device 10 and the magnetic flux density B_{1L}(y) caused by the magnetization of the shim member in the low-intensity magnetic field represented by the calculated value (step S150e). In other words, by employing the first calculating function 150e, the processing circuitry 150 calculates the third magnetic flux density B_{0L} representing the magnetic flux density based on the assumption that no shim member is present while the electric current having the first current value I_{L} is flowing through the static magnetic field magnet 101, on the basis of the first data and the magnetic characteristic of the shim members. More specifically, the processing circuitry calculates the value of the third magnetic flux density B_{0L} by calculating B_{0L}(y)=B_{L}(y)−B_{1L}(y) according to Expression (3).

Next, the third magnetic flux density B_{0L}(y) representing the magnetic flux density based on the assumption that no shim member is present while the electric current having the first current value I_{L} is flowing through the static magnetic field magnet 101 will be explained. The third magnetic flux density B_{0L}(y) is the magnetic flux density based on the assumption that no shim member is present while the electric current having the first current value I_{L} is flowing through the static magnetic field magnet 101. At first glance, it may seem possible to simply calculate the magnetic flux density B_{0L}(y) on the basis of the relationship between the value of the first current value I_{L} and the measuring position of the static magnetic field magnet 101 by using, for example, the Biot-Savart Law, or the like; however, in actuality, the static magnetic field magnet 101 has a complicated shape, and also, the static magnetic field magnet 101 itself may be slightly deformed by the magnetic field, for example. Consequently, a theoretical value obtained by a theoretical calculation according to the Biot-Savart Law or the like would not be able to serve as a sufficiently accurate value of the third magnetic flux density B_{0L}(y).

Accordingly, the processing circuitry 150 calculates the third magnetic flux density B_{0L}, by subtracting the magnetic flux density B_{1L}(y) roughly estimating the effect of the shim member from the first magnetic flux density B_{L}(y) represented by the measured value in the low-intensity magnetic field obtained by the measuring device 10. In other words, the third magnetic flux density B_{0L} is a value obtained by incorporating various realistic effects other than those eliminated as non-linear effects of the shim member, into the value of the magnetic flux density simply calculated by using the Biot-Savart Law on the basis of the value of the first current value I_{L}. Because these various effects are generally very small in quantity, it is possible to treat these effects as effects that are linear with respect to the first current value I_{L}.

After that, the processing circuitry 150 calculates the magnetic flux density B_{OR} directly caused by the static magnetic field magnet 101 on the assumption that no shim member is present in the rated-intensity magnetic field (with the second current value I_{R}), on the basis of the magnetic flux density B_{0L} directly caused by the static magnetic field magnet 101 on the assumption that no shim member is present in the low-intensity magnetic field (with the first current value I_{L}) (step S150f). More specifically, the processing circuitry 150 calculates the magnetic flux density B_{0R} by using the expression B_{0R}=I_{R}/I_{L}×B_{0L}, for example.

Further, at steps S150g through S150j, the processing circuitry 150 calculates a magnetic flux density B_{1R} caused by the magnetization of the shim members in the rated-intensity magnetic field. In this situation, based on the same concept as in Expression (3), Expression (5) presented below is obtained.

B_{R}(y)=B_{0R}(y)+B_{1R}(y) (5)

In Expression (5), the magnetic flux density B_{1R} denotes the magnetic flux density caused by the magnetic moment M_{R} that occurs in the shim members when an electric current having the second current value I_{R} is flowing through the static magnetic field magnet 101. In contrast, the magnetic flux density B_{0R} denotes the magnetic flux density based on the assumption that no shim member is present while an electric current having the second current value I_{R} larger than the first current value I_{L} is flowing through the static magnetic field magnet 101.

In the same manner as at step S150a, by employing the first calculating function 150e, the processing circuitry 150 calculates the magnetic field H_{R;shim} generated within the shim members by the static magnetic field magnet 101 in the rated-intensity magnetic field on the basis of the second current value I_{R} (step S150g). More specifically, by employing the first calculating function 150e, the processing circuitry 150 calculates the magnetic field H_{R;shim} generated within the shim members by using the Ampere's Law, the Biot-Savart Law, or the like, on the basis of the second current value I_{R}.

Subsequently, by employing the first calculating function 150e, the processing circuitry 150 calculates, on the basis of the magnetic characteristic of the shim members, the magnetic flux density B_{R;shim} within the shim members, on the basis of the magnetic field H_{R;shim} generated within the shim members and calculated at step S150g (step S150h).

More specifically, at step S150h, by employing the first calculating function 150e, the processing circuitry 150 calculates, as illustrated in FIG. 4, the point 32 that is the intersection point of the value (H_{R}) of the magnetic field H_{R};shim calculated at step S150h and the B-H curve 30 obtained from the memory 132 and further calculates the magnetic flux density B_{R;shim} within the shim members on the basis of the value B_{R} of the calculated point 32 on the vertical axis.

After that, by employing the first calculating function 150e, the processing circuitry 150 calculates the magnetic moment M_{R} of the shim members in the rated-intensity magnetic field, on the basis of the magnetic field H_{R;shim} generated within the shim members and the magnetic flux density B_{R;shim} within the shim members (step S150i). For example, by employing the first calculating function 150e, the processing circuitry 150 calculates the magnetic moment M_{R} of the shim members in the rated-intensity magnetic field by calculating M_{R}=B_{R}−μ₀H_{R} according to Expression (2).

Based on the same concept as in Expression (4), it is possible to express the magnetic flux density B_{1R} representing the magnetic flux density caused by the magnetic moment M_{R} that occurs in the shim member while the electric current having the second current value I_{R} is flowing through the static magnetic field magnet 101, by using Expression (6) presented below with the use of the information R(x,y) related to the positional arrangement of the shim member used in Expression (4).

B_{1R}(y)=∫d×M_{R}(x)R(x,y) (6)

By employing the first calculating function 150e and using Expression (6), the processing circuitry 150 calculates the magnetic flux density B_{1R} representing the magnetic flux density caused by the magnetization of the shim member in the rated-intensity magnetic field, on the basis of the magnetic moment M_{R} of the shim member in the rated-intensity magnetic field and the information R related to the positional arrangement of the shim member (step S150j). In other words, by employing the first calculating function 150e, the processing circuitry 150 calculates, by performing the series of processes at steps S150g to S150j, the magnetic flux density B_{1R} caused by the magnetic moment M_{R} that occurs in the shim member while the electric current having the second current value I_{R} is flowing through the static magnetic field magnet 101, on the basis of the information R related to the positional arrangement of the shim member and the magnetic characteristic of the shim member.

Subsequently, by using Expression (5), the processing circuitry 150 calculates the second magnetic flux density B_{R} representing the magnetic flux density in the rated-intensity magnetic field, by adding together the magnetic flux density B_{OR} that is directly caused by the static magnetic field magnet 101 in the rated-intensity magnetic field and was obtained at step S150f and the magnetic flux density B_{1R} that is caused by the magnetization of the shim member in the rated-intensity magnetic field and was obtained at step S150j (step S150k). In other words, by employing the first calculating function 150e, the processing circuitry 150 calculates the magnetic flux density B_{R} by adding the magnetic flux density B_{0R} to the magnetic flux density B_{1R}.

As explained above, by employing the first calculating function 150e, the processing circuitry 150 calculates, by performing the processes at steps S150a through S150k, the third magnetic flux density B_{0L} based on the assumption that no shim member is present while the electric current having the first current value I_{L} is flowing through the static magnetic field magnet 101, on the basis of the first data and the magnetic characteristic of the shim members. After that, by employing the first calculating function 150e, the processing circuitry 150 is able to calculate, even when the B-H curve is non-linear, for example, the second magnetic flux density B_{R} in the situation where the shim members are arranged with the first positional arrangement while the electric current having the second current value I_{R} larger than the first current value I_{L} is flowing through the static magnetic field magnet 101, on the basis of the calculated third magnetic flux density B_{0L} and the magnetic characteristic of the shim members.

Returning to the description of FIG. 2, the processing circuitry 150 calculates a positional arrangement of the shim members (information related to the positional arrangement of the shim members) in the rated-intensity magnetic field (step S160). In other words, by employing the second calculating function 150f, the processing circuitry 150 calculates a second positional arrangement with which the shim members are arranged, on the basis of the value of the second magnetic flux density B_{R} calculated at step S150. In other words, by employing the second calculating function 150f, the processing circuitry 150 calculates the positional arrangement of the shim members used for correcting uniformity of the static magnetic field, on the basis of second data.

In a specific example of a method for calculating the second positional arrangement, the processing circuitry 150 calculates, by employing the second calculating function 150f, the difference between an ideal distribution of magnetic flux density and the second magnetic flux density B_{R}, for example, and further expands the calculated difference between the magnetic flux density using a predetermined basis such as a spherical harmonic function, for example. The processing circuitry 150 calculates in which location and in what magnitude the magnetic moment should be present, on the basis of the type of the spherical harmonic function used for the expansion and the magnitude of an expansion coefficient and further calculates the second positional arrangement with which the shim members are arranged according to the result of the calculation.

Typically, the “information related to the positional arrangement of the shim members” denotes information related to positions of the shim members; however, possible embodiments are not limited to this example. For instance, the “information related to the positional arrangement of the shim members” may be information related to the quantity of the shim members. Further, for instance, the “information related to the positional arrangement of the shim members” may be information related to the magnitude and/or the direction of the magnetic moment of the shim members.

Subsequently, the processing circuitry 150 transmits and stores the second positional arrangement calculated at step S160 into the memory 132 (step S170). The second positional arrangement of the shim members being stored can be invoked by the processing circuitry 150 as necessary and may be used as an initial positional arrangement of the shim members, for example.

After that, the user arranges the shim members in the low-intensity magnetic field (step S180). The processing circuitry 150 stands by until the user finishes arranging the shim members. Because the process at step S180 is performed in the magnetic field having lower intensity than that of the rated-intensity magnetic field, the magnetic field attraction forces of the shim members are smaller compared to those in the rated-intensity magnetic field. The efficiency of the work is therefore enhanced.

Subsequently, by employing the receiver circuitry 110 and a measuring apparatus (not illustrated), for example, the MRI apparatus 100 measures the value of the magnetic flux density in the low-intensity magnetic field while the shim members are arranged with the second positional arrangement (step S190). In other words, by employing the acquiring function 150d, the processing circuitry 150 acquires data related to the magnetic flux density in the situation where the shim members are arranged with the second positional arrangement while the electric current having the first current value I_{L}, which is the current value corresponding to the low-intensity magnetic field, is flowing through the static magnetic field magnet 101.

After that, on the basis of the value of the magnetic flux density in the low-intensity magnetic field that corresponds to the situation where the shim members are arranged with the second positional arrangement and that was acquired by the acquiring function 150d at step S190, the processing circuitry 150 calculates a magnetic field uniformity value in the rated-intensity magnetic field in the situation where the shim members are arranged with the second positional arrangement (step S200). In this situation, as the magnetic field uniformity value resulting from the calculation, the processing circuitry 150 calculates a magnetic field uniformity value E_{R}(y) in each of different points of spatial positions, for example. In this situation, the letter y symbolically expresses the position vector of a three-dimensional position, for example, and is characterized with a set of polar coordinates such as (r,θ,φ) expressed by using the center of the magnetic field as the origin, for example. The magnetic field uniformity value E_{R}(y) is dependent on the second positional arrangement with which the shim members are arranged, via the magnetic moment M_{R} of the shim members.

Subsequently, the processing circuitry 150 judges whether or not the magnetic field uniformity values E_{R}(y) in the rated-intensity magnetic field calculated at step S200 satisfy a predetermined criterion (step S210). The criterion used for the judgment may be, for example, whether or not the variance among the magnetic field uniformity values E_{R}(y) is smaller than a predetermined value. When the magnetic field uniformity values E_{R}(y) calculated at step S200 do not satisfy the predetermined criterion (step S210: No), i.e., when the level of magnetic field uniformity is not sufficient in the situation where the shim members are arranged with the second positional arrangement, the positional arrangement is updated, and the process returns to step S150.

In this situation, for example, updating the positional arrangement denotes treating, at steps S150a through S150k, the situation where the shim members are arranged with the second positional arrangement as “the situation where the shim members are arranged with the first positional arrangement” in the processes at steps S150a through S150k described above. Further, for example, updating the positional arrangement denotes treating, at steps S150a through S150k, the value of the magnetic flux density measured at step S190 as the value of the first magnetic flux density B_{L} measured at step S140. Further, on the basis of step S150, a positional arrangement of the shim members in the rated-intensity magnetic field is newly calculated at step S160.

As explained herein, until the positional arrangement of the shim members satisfies the condition at step S210, the processes at steps S150 through S200 are repeatedly performed so as to update the positional arrangement of the shim members.

On the contrary, when the magnetic field uniformity values E_{R}(y) calculated at step S200 satisfy the predetermined criterion (step S210: Yes), the process proceeds to step S220. In other words, the static magnetic field power supply applies the rated-intensity magnetic field (step S220). More specifically, the static magnetic field power supply causes an electric current having the second current value I_{R}, which is the current value corresponding to the rated-intensity magnetic field, to flow through the static magnetic field magnet 101.

Subsequently, by employing the receiver circuitry 110 and a measuring apparatus (not illustrated), for example, the MRI apparatus 100 measures the value of the magnetic flux density in the rated-intensity magnetic field while the shim members are arranged with the second positional arrangement (step S230). In other words, by employing the acquiring function 150d, the processing circuitry 150 acquires data related to the magnetic flux density in the situation where the shim members are arranged with the second positional arrangement, while the electric current having the second current value I_{R}, which is the current value corresponding to the rated-intensity magnetic field, is flowing through the static magnetic field magnet 101.

After that, on the basis of the value of the magnetic flux density in the rated-intensity magnetic field that corresponds to the situation where the shim members are arranged with the second positional arrangement and that was acquired by the acquiring function 150d at step S230, the processing circuitry 150 calculates magnetic field uniformity values E_{R}(y) in the rated-intensity magnetic field in the situation where the shim members are arranged with the second positional arrangement (step S240). In this situation, for example, the letter y symbolically expresses the position vector of a three-dimensional position, for example, and is characterized with a set of polar coordinates such as (r,θ,φ) expressed by using the center of the magnetic field as the origin, for example.

Subsequently, the processing circuitry 150 judges whether or not the magnetic field uniformity values E_{R}(y) in the rated-intensity magnetic field calculated at step S240 satisfy a predetermined criterion (step S250). When the magnetic field uniformity values E_{R}(y) calculated at step S240 do not satisfy the predetermined criterion (step S250: No), i.e., when the positional arrangement theoretically predicted is not working well in actuality, the process returns to step S130, and the process is started over from the beginning. In that situation, the static magnetic field power supply decreases the magnetic field intensity from the level of the rated-intensity magnetic field to the level of the low-intensity magnetic field.

On the contrary, when the magnetic field uniformity values E_{R}(y) calculated at step S240 satisfy the predetermined criterion (step S250: Yes), it is determined that the shimming process has been completed, and the process is ended.

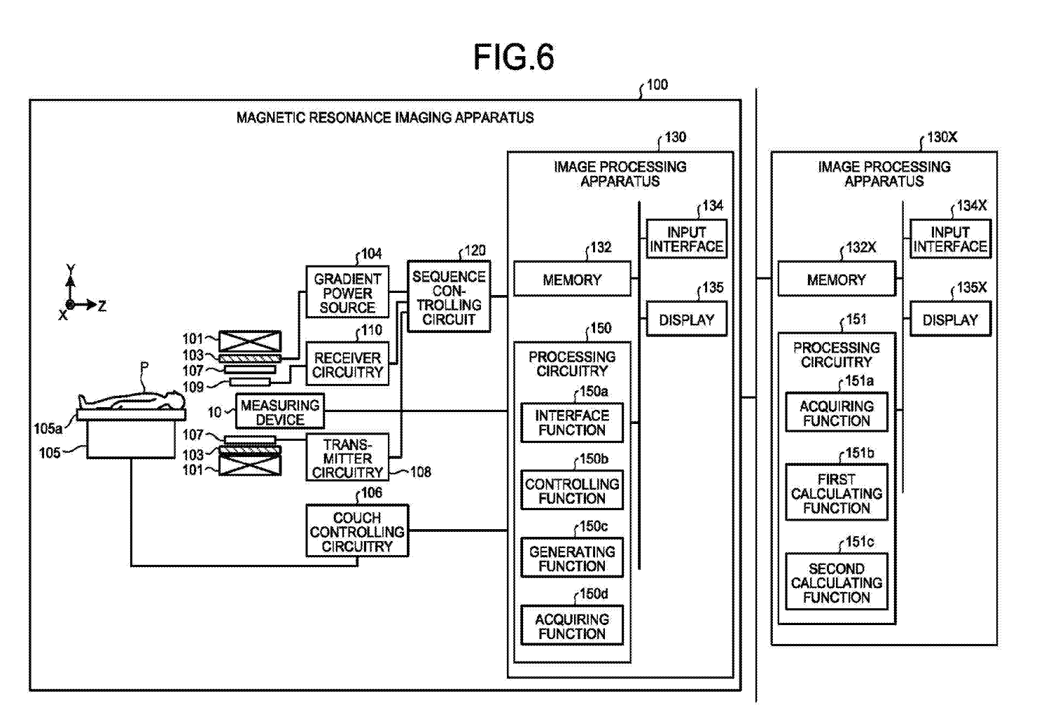

Possible embodiments are not limited to the embodiment described above. Although FIG. 1 illustrates the example where the image processing apparatus 130 is incorporated in the MRI apparatus 100, another arrangement is also acceptable in which, as indicated in FIG. 6, an image processing apparatus 130X independent of the MRI apparatus 100 is configured to perform the same processes as those performed by the image processing apparatus 130 illustrated in FIG. 1. In that situation, a memory 132X, processing circuitry 151, an input interface 134X, and a display 135X perform the same processes as those performed by the memory 132, the processing circuitry 150, the input interface 134, and the display 135 illustrated in FIG. 1, respectively. Further, an acquiring function 151a, a first calculating function 151b, and a second calculating function 151c, for example, have the same functions as those of the acquiring function 150d, the first calculating function 150e, and the second calculating function 150f, respectively.

Further, the example was explained in which, for instance, the processing circuitry 150 calculates the value of the magnetic field attraction force F(B_{L}) of the passive shim members in the low-intensity magnetic field at step S110; however, possible embodiments are not limited to this example. For instance, at step S110, the processing circuitry 150 may calculate the value of the magnetic field attraction force F(B_{R}) of the passive shim members in the rated-intensity magnetic field. In that situation, at step S120, the processing circuitry 150 may judge whether or not the magnetic field attraction force F(B_{R}) meets the safety standard and may further judge whether or not the processes at step S130 and thereafter should be performed on the basis of the result of the judgment.

The example was explained in which, at step S130, the shim members are arranged with the first positional arrangement, which is the predetermined positional arrangement, for example; however, possible embodiments are not limited to this example. For instance, the shim members do not necessarily have to be in the arranged state at step S130. In that situation, at step S130, the static magnetic field power supply applies, to the static magnetic field magnet 101, an electric current having the first current value I_{L} corresponding to the low-intensity magnetic field (the first magnetic flux density B_{L}) having the value set at step S100, while no shim member is arranged. By employing the acquiring function 150d, the processing circuitry 150 acquires, at step S140, the first data related to the first magnetic flux density B_{L} in the situation where the electric current having the first current value I_{L} is flowing through the static magnetic field magnet 101 while no shim member is arranged. At step S150, by employing the first calculating function 150e, the processing circuitry 150 calculates the second magnetic flux density B_{R} in the situation where the electric current having the second current value I_{R} (the current value substantially corresponding to the rated-intensity magnetic field) larger than the first current value I_{L} is flowing through the static magnetic field magnet 101. At step S160, by employing the second calculating function 150f, the processing circuitry 150 calculates a positional arrangement of the shim members used for correcting the uniformity of the static magnetic field generated by the static magnetic field magnet 101, on the basis of the value of the second magnetic flux density B_{R} calculated at step S150.

Further in the embodiment, the example was explained in which, at step S150a, by employing the first calculating function 150e, the processing circuitry 150 calculates the value of the third magnetic flux density B_{0L} representing the magnetic flux density directly caused by the static magnetic field magnet 101 in the low-intensity magnetic field, on the basis of the first current value I_{L}; however, possible embodiments are not limited to this example. For instance, the MRI apparatus 100 may calculate the third magnetic flux density B_{0L} by measuring a magnetic flux density in the situation where no shim member is arranged, by employing the receiver circuitry 110 or a measuring apparatus (not illustrated). In that situation, by employing the acquiring function 150d, the processing circuitry 150 further acquires data related to the magnetic flux density Bₓ in the situation where no shim member is arranged while a predetermined electric current Iₓ is flowing through the static magnetic field magnet 101. By employing the first calculating function 150e, the processing circuitry 150 calculates the second magnetic flux density B_{R}, on the basis of the first data, the second data, and a magnetic characteristic of the shim members.

In this situation, when the predetermined current Iₓ has a value equal to the first current value I_{L}, the third magnetic flux density B_{0L} is equal to the magnetic flux density Bₓ. In that situation, at step S150e, the processing circuitry 150 uses the value of the magnetic flux density Bₓ as the value of the third magnetic flux density B_{0L}, instead of calculating the third magnetic flux density B_{0L}.

In contrast, when the predetermined current Iₓ has a value different from the first current value I_{L}, the processing circuitry 150 calculates, at step S150e, the value of the third magnetic flux density B_{0L} from the expression B_{0L}=Bₓ×I_{L}/Iₓ.

The processes at steps S150a through S150f and the processes at steps S150g through S150j may be performed in parallel with each other. Accordingly, for example, the processing circuitry 150 may perform the processes at steps S150a through S150f after performing the processes at steps S150g through S150j.

In the embodiment, the example was explained in which the hysteresis is low; however, possible embodiments are not limited to this example. The embodiment is similarly applicable to situations where hysteresis occurs during the magnetization process and/or the demagnetization process. Further, although the example with the passive shimming process was explained in the embodiment, possible embodiments are not limited to this example. For instance, the embodiment is similarly applicable to situations where an active shimming process is performed.

A Magnetic Field Adjusting Apparatus

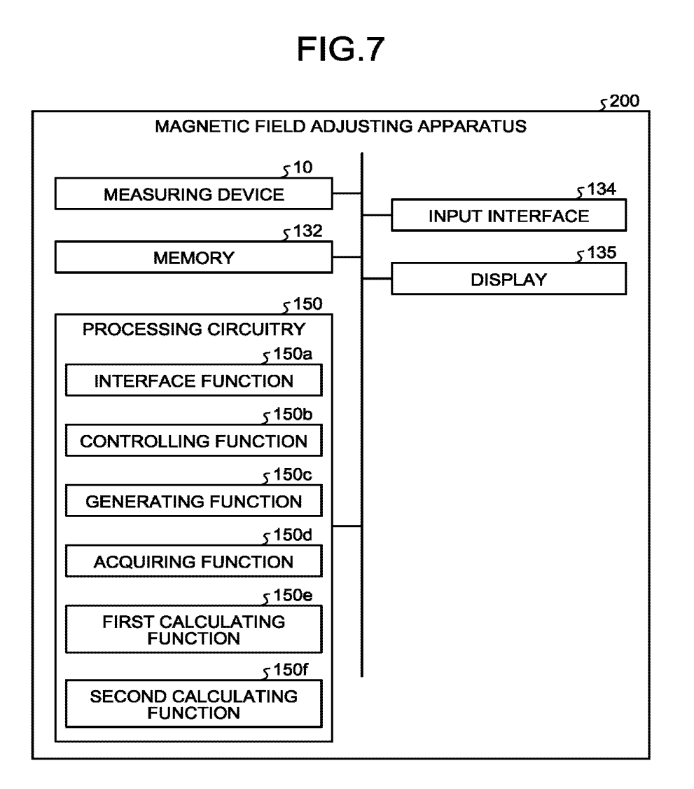

Further, in the embodiment, the example was explained in which the MRI apparatus 100 performs the magnetic field adjusting process described above; however, possible embodiments are not limited to this example. For instance, as illustrated in FIG. 7, a magnetic field adjusting apparatus 200 provided independently of the MRI apparatus 100 may perform the magnetic field adjusting process described above. The functions in the blocks illustrated in FIG. 7 have the same functions as those illustrated in the blocks illustrated in FIG. 1 and referred to by using the same reference characters.

The magnetic field adjusting apparatus 200 includes the processing circuitry 150 configured to calculate a positional arrangement of the shim members used for correcting uniformity of the static magnetic field, on the basis of the data related to a static magnetic field acquired while the static magnetic field magnet is generating the static magnetic field having magnetic field intensity lower than the rated magnetic field intensity required by imaging processes performed by the MRI apparatus.

Further, by employing the measuring device 10, the magnetic field adjusting apparatus 200 is configured to acquire the first data related to the static magnetic field while the static magnetic field magnet is generating the static magnetic field having magnetic field intensity lower than the rated magnetic field intensity required by imaging processes performed by the MRI apparatus. Further, by employing the processing circuitry 150, the magnetic field adjusting apparatus 200 is configured to calculate the positional arrangement of the shim members used for correcting the uniformity of the static magnetic field, on the basis of the first data.

Computer Programs

Further, the instructions described in the processing procedures explained in the embodiment above may be executed on the basis of a computer program (hereinafter, “program”) configured as software. It is possible to achieve the same effects as those achieved by the MRI apparatus 100 described in the embodiment above, by arranging a generic computer to store the program therein and to read the stored program. The instructions described in the embodiment above may be recorded as a computer-executable program onto a magnetic disk (a flexible disk, a hard disk, or the like), an optical disk (a Compact Disk Read-Only Memory [CD-ROM], a Compact Disk Recordable [CD-R], a Compact Disk Rewritable [CD-RW], a Digital Versatile Disk Read-Only Memory [DVD-ROM], a DVD Recordable [DVD±R], DVD Rewritable [DVD±RW], or the like), a semiconductor memory, or a similar recording medium. The program may be stored in any format, as long as the computer or an incorporated system is able to read the program from the storage medium. When the computer reads the program from the recording medium and causes a Central Processing Unit (CPU) to execute the instructions described in the program on the basis of the program, the computer is able to realize the same operations as those performed by the MRI apparatus 100 described in the embodiment above. Further, when obtaining or reading the program, the computer may obtain or read the program via a network.

Furthermore, any part of the processes to realize the embodiment described above may be executed on the basis of the instructions in the program installed from the storage medium into a computer or an incorporated system, by an Operating System (OS) working in the computer, database management software, or middleware (MW) such as a network. Further, the storage medium does not necessarily have to be a medium provided independently of the computer or the incorporated system; the storage medium may be a storage medium that downloads and stores therein or temporarily stores therein the program transferred via a Local Area Network (LAN), the Internet, or the like. Further, the quantity of the storage medium does not necessarily have to be one; possible examples of the storage medium according to the embodiment include the situation where the processes described in the embodiment above are executed from a plurality of media. The configurations of the one or more media are not particularly limited.

The computer or the incorporated system according to the embodiment is configured to execute the processes described in the embodiment above, on the basis of the program stored in the one or more storage media and may be structured with a single apparatus such as a personal computer or a microcomputer or may be structured with a system in which a plurality of apparatuses are connected together via a network. Further, the computer according to the embodiment does not necessarily have to be a personal computer and may be an arithmetic processing unit included in an information processing apparatus, a microcomputer, or the like. The term “computer” generally refers to any device or apparatus capable of realizing the functions described in the embodiment by using the program.

According to at least one aspect of the embodiments described above, it is possible to perform the shimming process efficiently.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic field adjusting method comprising:
acquiring, by using a measuring device, first data related to a static magnetic field while a static magnetic field magnet is generating a static magnetic field having magnetic field intensity lower than rated magnetic field intensity required by an imaging process performed by a magnetic resonance imaging apparatus; and
calculating, by using processing circuitry, a positional arrangement of a shim member used for correcting uniformity of a static magnetic field on a basis of the first data.

2. The magnetic field adjusting method according to claim 1, wherein the first data is data that is related to a magnetic flux density.

3. The magnetic field adjusting method according to claim 1, wherein the positional arrangement of the shim member is calculated by the processing circuitry on a basis of second data, the second data being calculated from the first data while using a magnetization curve of a substance contained in the shim member and the second data being related to a static magnetic field in a situation where the static magnetic field magnet is generating a rated-intensity static magnetic field.

4. The magnetic field adjusting method according to claim 1, wherein
the first data is data related to a first magnetic flux density in a situation where an electric current having a first current value is flowing through the static magnetic field magnet,
the calculating of the positional arrangement of the shim member includes:
calculating, by using the processing circuitry, a second magnetic flux density in a situation where an electric current having a second current value larger than the first current value is flowing through the static magnetic field magnet, on a basis of the first data; and
calculating, by using the processing circuitry, the positional arrangement of the shim member on a basis of a value of the second magnetic flux density.

5. The magnetic field adjusting method according to claim 1, wherein
the first data is data related to a first magnetic flux density in a situation where the shim member is arranged with a first positional arrangement while an electric current having a first current value is flowing through the static magnetic field magnet, and
the calculating of the positional arrangement of the shim member includes calculating, by using the processing circuitry, a value of a second magnetic flux density in a situation where the shim member is arranged with the first positional arrangement while an electric current having a second current value larger than the first current value is flowing through the static magnetic field magnet, on a basis of the first data.

6. The magnetic field adjusting method according to claim 5, wherein the calculating of the positional arrangement of the shim member includes calculating, by using the processing circuitry, the value of the second magnetic flux density on a basis of the first data and a magnetic characteristic of the shim member.

7. The magnetic field adjusting method according to claim 5, wherein the calculating of the positional arrangement of the shim member includes calculating, by using the processing circuitry, a second positional arrangement with which the shim member is arranged, on a basis of the value of the second magnetic flux density.

8. The magnetic field adjusting method according to claim 6, wherein the magnetic characteristic is a correspondence relationship between a magnitude of a magnetic field applied to the shim member and a magnitude of a magnetic flux density caused on the shim member.

9. The magnetic field adjusting method according to claim 6, wherein the magnetic characteristic is magnetic permeability of the shim member expressed as a mathematical function of at least one selected from between magnetic field and magnetic flux density.

10. The magnetic field adjusting method according to claim 6, wherein the calculating of the positional arrangement of the shim member includes: calculating, by using the processing circuitry, a third magnetic flux density representing a magnetic flux density in a situation where the shim member is not present while an electric current having the first current value is flowing through the static magnetic field magnet, on a basis of the first data and the magnetic characteristic of the shim member; and further calculating the value of the second magnetic flux density on a basis of the calculated third magnetic flux density and the magnetic characteristic of the shim member.

11. The magnetic field adjusting method according to claim 5, further comprising:
acquiring, by using the measuring device, second data related to a magnetic flux density in a situation where the shim member is not arranged while an electric current having a predetermined current value is flowing through the static magnetic field magnet, wherein
the calculating of the positional arrangement of the shim member includes calculating, by using the processing circuitry, the value of the second magnetic flux density on a basis of the first data, the second data, and a magnetic characteristic of the shim member.

12. The magnetic field adjusting method according to claim 6, wherein the calculating of the positional arrangement of the shim member includes, by using the processing circuitry, correcting the magnetic characteristic on a basis of the first data and calculating the value of the second magnetic flux density on a basis of the corrected magnetic characteristic.

13. A magnetic field adjusting apparatus comprising: processing circuitry configured to calculate a positional arrangement of a shim member used for correcting uniformity of a static magnetic field on a basis of data that is related to a static magnetic field and is acquired while a static magnetic field magnet is generating a static magnetic field having magnetic field intensity lower than rated magnetic field intensity required by an imaging process performed by a magnetic resonance imaging apparatus.

14. A magnetic resonance imaging apparatus comprising: processing circuitry configured to calculate a positional arrangement of a shim member used for correcting uniformity of a static magnetic field on a basis of data that is related to a static magnetic field and is acquired while a static magnetic field magnet is generating a static magnetic field having magnetic field intensity lower than a rated magnetic field intensity required by an imaging process performed by the magnetic resonance imaging apparatus.
